(19) United States
(12) Patent Application Publication
(10) Pub. No.: US 2020/0248139 A1
(43) Pub. Date: Aug. 6, 2020

(54) METHODS FOR GENERATING AND USING ORGANOIDS AND CELLS THEREOF

(71) Applicant: REGENTS OF THE UNIVERSITY OF MINNESOTA, Minneapolis, MN (US)

(72) Inventors: Timothy D. O'Brien, Cokato, MN (US); Beth A. Lindborg, St. Paul, MN (US); Amanda Vegoe, Apple Valley, MN (US); Jakub Tolar, Minneapolis, MN (US)

(21) Appl. No.: 16/637,450

(22) PCT Filed: Aug. 8, 2018
(86) PCT No.: PCT/US2018/045775

§ 371 Date: Feb. 7, 2020

Related U.S. Application Data
(60) Provisional application No. 62/542,669, filed on Aug. 8, 2017.

(51) Int. Cl. C12N 5/079; A61K 35/30

(52) U.S. Cl. CPC C12N 5/0618; C12N 2513/00; C12N 2501/905; A61K 35/30

(57) ABSTRACT

This disclosure describes methods for organoid generation including, for example, for generation of a mid-brain organoid including an A9 neuron. Specifically, the methods comprising: introducing an input cell into a cell culture medium comprising hyaluronic acid, wherein the input cell comprises an embryonic stem cell, an induced pluripotent stem cell, or a neural progenitor cell; transferring the input cell to a cell culture device; and culturing the cell in the cell culture device for at least 7 days. This disclosure further describes methods for using the organoids.

### CONTINUING APPLICATION DATA

This application claims the benefit of U.S. Provisional Application Ser. No. 62/542,669, filed Aug. 8, 2017, which is incorporated by reference herein.

### BACKGROUND

Pluripotent stem cells (PSCs), such as induced pluripotent stem cells (iPSCs) or embryonic stem cells (ESCs), can self-organize under various conditions to form complex tissue structures—also known as organoids—that recapitulate important developmental features and structural and functional characteristics typical of particular tissues. The cellular and structural complexity of these organoids and their fidelity to the structure of corresponding tissues in vivo help make them readily accessible in vitro models for a wide range of physiologic and metabolic studies, for pharmaceutical screens, and as models for human pathological conditions.

### SUMMARY OF THE INVENTION

This disclosure describes methods for organoid generation including, for example, for generation of a mid-brain organoid including an A9 neuron (also referred to herein as an A9 type nigral dopaminergic neuron, an A9 nigral dopaminergic neuron, or an A9 dopaminergic neuron).

In one aspect, this disclosure describes a method that includes: introducing an input cell into a cell culture medium comprising hyaluronic acid; transferring the input cell to a cell culture device; culturing the cell in the cell culture device for at least 7 days; and producing a midbrain organoid comprising an A9 neuron.

In some embodiments, the input cell includes an embryonic stem cell, an induced pluripotent stem cell, or a neural progenitor cell.

In some embodiments, the method includes removing the input cell from a culture plate. Removing the input cell from the culture plate may include, for example, exposing the cell to at least one of a cell dissociation enzyme, a citrate buffer, phosphate buffered saline, and a cell culture media.

In some embodiments, introducing an input cell into a cell culture medium includes introducing the cell into a cell culture matrix. Introducing the cell into the cell culture matrix may include, for example, introducing a single cell, introducing a colony of cells, or introducing an embryoid body.

In some embodiments, transferring the input cell to a cell culture device includes transferring the cell in the cell culture matrix. In some embodiments, at the time of transferring the input cell to a cell culture device, the cell culture matrix may include sections of up to 80 of up to 50 of up to 25 of up to 15 or of up to 10 μL. In some embodiments, at the time of transferring the input cell to a cell culture device, the cell culture matrix may include sections of at least 1 μL. In some embodiments, at the time of transferring the input cell to a cell culture device, the input cell is present in the cell culture matrix at a concentration of at least 7.6×10⁵ cells per 10 μL matrix, at least 1.2×10⁶ cells per 10 μL matrix, or at a concentration of at least 1.4×10⁶ cells per 10 μL matrix. In some embodiments, at the time of transferring the input cell to a cell culture device, the input cell is present in the cell culture matrix at a concentration of up to 3×10⁶ cells per 10 μL matrix.

In some embodiments, the cell culture device includes a second cell culture medium. The second cell culture medium may include, in some embodiments, a serum-free cell culture medium, a feeder-free cell culture medium, an iPSC medium, and/or a neural medium. The second cell culture medium may include, in some embodiments, a neural induction factor, a neural growth factor, or both. The neural induction factor and/or the neural growth factor may include, for example, at least one of N2, B27, FGF2, TGFβ, insulin, ascorbate, and glutamate.

In some embodiments, the cell culture device may include a bioreactor. In some embodiments, the cell culture device may include a gas permeable membrane surface and/or a silicone surface. In embodiments, wherein the cell culture device includes a silicone surface, the silicone surface can include dimethyl silicone. In some embodiments, wherein the cell culture device includes a gas permeable membrane surface, the method may further include removing the cell from the gas permeable membrane surface.

In some embodiments, culturing the cell in the cell culture device includes culturing the cell at room temperature. In some embodiments, culturing the cell in the cell culture device includes culturing the cell at 37° C. In some embodiments, culturing the cell in the cell culture device includes culturing the cell in hypoxic conditions.

In some embodiments, wherein the method includes introducing the cell into a cell culture matrix the method may further include removing the cell culture matrix from the midbrain organoid. The cell culture matrix may be removed using a mechanical method and/or an enzymatic method.

In some embodiments, the method includes dis-aggregating the cells of the midbrain organoid to produce a population of individualized cells. In some embodiments, the method may also include culturing a cell from the population of individualized cells.

In some embodiments, the midbrain organoid includes at least one of a cell expressing glial fibrillary acidic protein (GFAP); a cell expressing microtubule associated protein 2 (MAP2); and a cell expressing myelin basic protein (MBP).

In some embodiments, the midbrain organoid includes at least one of an oligodendrocyte, an astrocyte, and a polydendrocyte.

In some embodiments, the A9 neuron may be characterized by expression of tyrosine hydroxylase and Girk2.

In some embodiments, the midbrain organoid includes a cell exhibiting expression of at least one of nucleostemin (GNL3), SOX1, SOX2, β-3 tubulin (TUBB3), and nestin (NES). In some embodiments, the midbrain organoid includes a cell exhibiting expression of at least one of nuclear receptor subfamily 4 group A member 2 (NR4A2); LIM homeobox transcription factor 1 alpha (LMX1A); forkhead Box A2 (FOXA2); and orthodenticle homeobox 2 (OTX2). In some embodiments, the midbrain organoid includes a cell exhibiting expression of at least one of tyrosine hydroxylase (TH); torsin family 1 member A (TOR1A); corin, serine peptidase (CORIN); and dopa decarboxylase (DDC). In some embodiments, the midbrain organoid includes a cell exhibiting expression of potassium voltage-gated channel subfamily J member 6 (KCNJ6). In some embodiments, the midbrain organoid includes a cell exhibiting expression of calbindin 1 (CALB1). In some embodiments, the midbrain organoid includes an A10 neuron. The A10 neuron may be characterized by expression of at least one of tyrosine hydroxylase, calbindin 1 (CALB1), and Nurr1. In some embodiments, the expression includes gene expression. In some embodiments, the expression includes protein expression.

In another aspect, this disclosure describes a midbrain organoid generated using the methods described herein and methods of using that midbrain organoid. For example, the midbrain organoid may be used as a source of therapeutic cells for the treatment of a brain disorder or in a model of a brain disorder.

In a further aspect, this disclosure describes an A9 neuron generated using the methods of described herein and methods of using that A9 neuron.

As used herein, an “organoid” contains an organ-specific cell type, is capable of recapitulating a specific function of the organ, and contains a cell and/or structure that is spatially organized similar to that organ.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (for example, 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1 shows cerebral organoids from iPSC lines CS1 and CBB after 8.5-14 weeks of culture in ESSENTIAL 8 medium in GREX 100 cell culture devices. A) Immunohistochemical stains on histologic sections of a large organoid derived from cell line CBB show markers of differentiation to astrocytes (GFAP, glial fibrillar acidic protein), neurons (MAP2, microtubule-associated protein), and oligodendrocytes (MBP, myelin basic protein) in extensive regions. Size bar in first panel=5787 B) Immunohistochemical stains as in panel A on histologic sections of organoids from CS1 and CBB cell lines (15-343=CS1 13 weeks, 16-043=CBB 14 weeks, and 16-009=CBB 8.5 weeks) showing high magnification detail of astrocytes, neurons, and oligodendrocytes. Size bars=50 μm.

FIG. 2 shows exemplary histology of midbrain organoids. Upper panels: Left image shows confocal microscopy of histologic sections of a midbrain organoid derived from iPSC cell line CBB. Cells were cultured for 14 weeks in a GREX 100 cell culture device. Immunofluorescent double labeling for tyrosine hydroxylase (TH) and G Protein-Activated Inward Rectifier Potassium Channel 2 (Girk2) shows their co-localization (arrows), confirming the presence of A9 type nigral dopaminergic neurons in the organoid. Right image shows control staining for TH and Girk2 double labeling in neurons in the substantia nigra of a normal baboon brain. Lower Panels: Immunohistochemical labeling of adjacent histologic sections of a midbrain organoid (derived from the CBB cell line and cultured for 8.5 weeks) showing tyrosine hydroxylase and Girk2 labeling in neurons consistent with A9 dopaminergic neurons.

FIG. 3 shows immunohistochemical staining of cerebral organoids derived from cell lines CS1 (13 weeks in GREX 100 cell culture device) and CBB (8.5 weeks & 14 weeks in GREX 100 cell culture device). In all three conditions, positive nuclear staining was seen for Nurr1, a marker of dopaminergic neuron precursors, and cytoplasmic labeling was present for tyrosine hydroxylase and Girk2, markers of A9 nigral dopaminergic neurons. None of the conditions showed positive staining for calbindin which is a marker of A10 dopaminergic neurons.

FIG. 4 shows expression of neural stem/progenitor cell markers in exemplary cerebral organoids. RNA-Seq gene expression analysis of pooled, 5-6 week cerebral organoids derived from 2 different iPSC lines (1024 & R76) with 3 biological replicates of each. The panel shows moderate to high expression of genes that are markers of neural stem/progenitor cells (GNL3, nucleostemin; SOX1; SOX2; TUBB3, β-3 tubulin; and NES, nestin)) and shows a consistent pattern of expression of these genes among the replicates. AXL codes for the receptor protein for the Zika virus.

FIG. 5 shows expression of brain cell-type markers in exemplary cerebral organoids. RNA-Seq gene expression analysis of pooled, 5-6 week cerebral organoids derived from 2 different iPSC lines (1024 & R76) with 3 biological replicates of each. The panel provides evidence for the presence of neurons (indicated by the expression levels of DCX, doublecortin; RELN, reelin; MAP2, microtubule associated protein 2; and SYP, synaptophysin), oligodendrocytes (indicated by the expression levels of MBP, myelin basic protein; OLIG2, oligodendrocyte lineage transcription factor 2), astrocytes (indicated by the expression levels of GFAP, glial fibrillar acidic protein; SLC1A3, solute carrier family 1 member 3), and polydendrocytes (indicated by the expression levels of CSPG4, chondroitin sulfate proteoglycan 4). This mixture of cell types mirrors the patterns of cell populations in normal human brain.

FIG. 6 shows expression of dopaminergic neuron markers in exemplary cerebral organoids. RNA-Seq gene expression analysis of pooled, 5-6 week cerebral organoids derived from 2 different iPSC lines (1024 & R76) with 3 biological replicates of each. The panel provides evidence for the presence of dopaminergic neuron progenitors (indicated by the expression levels of NR4A2, nuclear receptor subfamily 4 group A member 2; LMX1A, LIM homeobox transcription factor 1 alpha; FOXA2, forkhead Box A2; and OTX2, orthodenticle homeobox 2), dopaminergic neurons (indicated by the expression levels of TH, tyrosine hydroxylase; TOR1A, torsin family 1 member A; CORIN, corin, serine peptidase; and DDC, dopa decarboxylase), A9 nigral dopaminergic neurons (indicated by the expression levels of KCNJ6, potassium voltage-gated channel subfamily J member 6), and A10 dopaminergic neurons (indicated by the expression levels of CALB1, calbindin 1).

FIG. 7 shows expression of transplant engraftment success markers in exemplary cerebral organoids. RNA-Seq gene expression analysis of pooled, 5-6 week cerebral organoids derived from 2 different iPSC lines (1024 & R76) with 3 biological replicates of each. The panel provides evidence for the presence of moderate to high levels of gene markers associated with positive engraftment outcomes in a rodent model of Parkinson's disease (PD) (EN1, engrailed homeobox 1; EN2, engrailed homeobox 2; PAX8, paired box 8; ETV5, ETS variant 5; SPRY1, Sprouty RTK signaling antagonist 1; CNPY1, canopy FGF signaling regulator 1; WNT1, Wnt family member 1; and FGF8, fibroblast growth factor 8) and very low to moderate levels of expression of genes associated with negative engraftment outcomes in a rodent model of PD (EPHA3, EPH receptor A3; FEZF1, FEZ family zinc finger 1; and WNT7B, Wnt family member 7B).

FIG. 8 shows expression of brain regional markers in exemplary cerebral organoids. RNA-Seq gene expression analysis of pooled, 5-6 week cerebral organoids derived from 2 different iPSC lines (1024 & R76) with 3 biological replicates of each. The panel provides evidence for very low levels of gene markers associated with forebrain development (PAX6, paired box 6; FOXG1, forkhead box G1; SIX3, SIX homeobox 3), very low levels of rostral diencephalic markers (BARHL1, BarH like homeobox 1; and BARHL2, BarH like homeobox 2), very low levels of markers for rostral midbrain (DBX1, developing brain homeobox 1; WNT8B, Wnt family member 8B; NKX2-1 NK2 homeobox 1; NKX2-1-AS1, NKX2-1 antisense RNA 1; NKX2-2, NK2 homeobox 2; NKX2-3, NK2 homeobox 3; NKX2-4, NK2 homeobox 4; and PITX2, paired like homeodomain 2), and low to moderate levels of markers for hindbrain (HOXA2, homeobox A2; ISL1, ISL LIM homeobox 1, and EGR2, early growth response 2).

FIG. 9 shows expression of markers for non-dopaminergic neuron types in exemplary cerebral organoids. RNA-Seq gene expression analysis of pooled, 5-6 week cerebral organoids derived from 2 different iPSC lines (1024 & R76) with 3 biological replicates of each. The panel provides evidence for the presence of low levels of a marker for cholinergic neurons (CHAT, choline o-acetyltransferase), very low to absent levels of markers for serotonergic neurons (SLC6A4, solute carrier family 6 member 4; TPH1, tryptophan hydroxylase 1; and TPH2, tryptophan hydroxylase 2), low to moderate levels of markers for glutaminergic neurons (SLC17A7, solute carrier family 17 member 7; and SLC17A6, solute carrier family 17 member 6) and moderate levels of a GABAergic neuron marker (SLC6A1, solute carrier family 6 member 1).

FIG. 10 shows expression of markers of neuron subtypes in exemplary midbrain organoids. RNA-Seq gene expression analysis of pooled, 5-6 week cerebral organoids derived from 2 different iPSC lines (1024 & R76) with 3 biological replicates of each. The panel provides evidence for the preponderance of dopaminergic neuron markers versus other neuronal subtypes in organoids, consistent with a midbrain phenotype. Specifically, there is expression of dopaminergic neuron progenitors (NR4A2, nuclear receptor subfamily 4 group A member 2; LMX1A, LIM homeobox transcription factor 1 alpha; FOXA2, forkhead Box A2; and OTX2, orthodenticle homeobox 2), dopaminergic neurons (TH, tyrosine hydroxylase; TOR1A, torsin family 1 member A; CORIN, corin, serine peptidase; and DDC, dopa decarboxylase), A9 nigral dopaminergic neurons (KCNJ6, potassium voltage-gated channel subfamily J member 6), and A10 dopaminergic neurons (CALB1, calbindin 1). There is also evidence for the presence of low levels of a marker for cholinergic neurons (CHAT, choline o-acetyltransferase), very low to absent levels of markers for serotonergic neurons (SLC6A4, solute carrier family 6 member 4; TPH1, tryptophan hydroxylase 1; and TPH2, tryptophan hydroxylase 2), low to moderate levels of markers for glutaminergic neurons (SLC17A7, solute carrier family 17 member 7; and SLC17A6, solute carrier family 17 member 6) and moderate levels of a GABAergic neuron marker (SLC6A1, solute carrier family 6 member 1).

FIG. 11(A-C) shows an exemplary patch-clamp study of neuronal electrophysiology of neurons derived from an organoid derived as described in Example 2. FIG. 11A shows current injections evoked action potentials with stable resting membrane potential. FIG. 11B shows an exemplary trace of a cell with spontaneous synaptic activity (likely a mEPSC) in voltage clamp. FIG. 11C shows an exemplary response to 10 μM NMDA, indicating the presence of glutaminergic neurons.

FIG. 12 shows exemplary tissue sections. Nude rat brain (striatum) was transplanted with 300,000 cells derived from 8-week organoids. Four months later, tissue sections were prepared. Immunohistochemistry using a human-specific STEM121 monoclonal antibody demonstrated robust engraftment of human cells four months post-transplantation.

### DETAILED DESCRIPTION OF ILLUSTRATIVE EMBODIMENTS

This disclosure describes methods for organoid generation including, for example, for generation of a mid-brain organoid including an A9 neuron.

In contrast to previously published methods for creating cerebral organoids which generated organoids that exhibited certain features indicating early forebrain, midbrain, and hindbrain differentiation (see, e.g., Lindborg et al. Stem Cells Translational Medicine, 2016, 5(7):970-979), the methods described herein induce specification to predominantly one brain region. Using the methods described herein to generate organoids, RNA-Seq analysis of gene expression detected negligible levels of forebrain markers and low levels of hindbrain markers and a predominance of midbrain markers. The methods described herein may also, in some embodiments, simultaneously allow the development of other critical brain cells including astrocytes, oligodendrocytes, and polydendrocytes without the use of specific brain induction factors.

In one aspect, this disclosure describes a method that includes: introducing an input cell into a cell culture medium including hyaluronic acid; transferring the input cell to a cell culture device; and culturing the cell in the cell culture device for at least 7 days. The method produces a midbrain organoid comprising an A9 neuron.

### Input Cells

An input cell may include, for example, an embryonic stem cells (ESC), an induced pluripotent stem cell (iPSC), or a neural progenitor cell. An ESC may include, for example, an H9 cell. An iPSC may include an iPSC cell line including. In some embodiments, an iPSC cell line may include a cell line of Table 1. In some embodiments, an iPSC cell line may include CS1, CBB, 1024, or R76.

In some embodiments, the method may include preparing the input cell and/or removing the input cell from a culture plate. Cells may be removed from a culture plate by any suitable method. For example, the cell may be exposed to at least one of a cell dissociation enzyme, a collagenase, a citrate buffer, phosphate buffered saline (PBS), and a cell culture media. In some embodiments, the cell may be exposed to a cell passaging solution. A cell dissociation enzyme may include, for example, a collagenase, a catalase, a dispase, an elastase, a hyaluronidase, papain, a trypsin, TrypLE (Thermo Fisher Scientific, Waltham, Mass.,) ACCUMAX (Sigma-Aldrich, St. Louis, Mo.), ACCUTASE (Sigma-Aldrich, St. Louis, Mo.), etc.

Introducing the Cell into Cell Culture Medium

The method for organoid generation includes introducing the input cell into a cell culture medium including hyaluronic acid. In some embodiments, the cell culture medium also includes chitosan.

In some embodiments, the cell culture medium is a solution. In some embodiments, the hyaluronic acid of the cell culture media may be bonded to a surface.

In some embodiments, the cell culture medium preferably includes a cell culture matrix. In some embodiments, the cell culture matrix includes a hydrogel. In some embodiments, a cell culture medium includes Cell-Mate3D (BRTI Life Sciences, Two Harbors, Minn.). In some embodiments, introducing the input cell into a cell culture matrix include embedding the cell in the cell culture matrix.

An input cell may be introduced into the cell culture medium as a single cell, as a colony of cells, as a group of cells, or as a sphere including, for example, as an embryoid body.

Cells may be introduced into the cell culture medium at any suitable concentration. In some embodiments, including for example, when the cell culture medium includes a cell culture matrix, the input cell may be present in the cell culture matrix at a concentration of at least 7.6×10⁵ cells per 10 μL matrix, at least 1.2×10⁶ cells per 10 μL matrix, at least 1.4×10⁶ cells per 10 μL matrix, or at least 1.6×10⁶ cells per 10 μL matrix. In some embodiments, the input cell may be present in the cell culture matrix at a concentration of up to 3×10⁶ cells per 10 μL matrix.

Without wishing to be bound by theory, it is believed that introducing the cells into a cell culture matrix at a very high concentration (for example, at least 7.6×10⁵ cells per 10 μL matrix) results in the formation of a matrix with low integrity. For example, at 7.6×10⁵ cells per 10 μL matrix, cells are present at twice the concentration used in Lindborg et al. Stem Cells Translational Medicine, 2016, 5(7):970-979, and the matrix demonstrates significantly less integrity. The resulting low integrity construct dissociates after addition to the cell culture device, yet, despite the loss of three-dimensional structure, organoid formation is improved over the use of cell culture matrix with lower concentrations of cells. Moreover, as described below, disintegration of the matrix may allow the resulting organoids to emerge from the matrix without manual removal.

### Cell Culture Device

After the input cell has been introduced into a cell culture medium including hyaluronic acid, the cell is transferred to a cell culture device.

In some embodiments, transferring the input cell to a cell culture device includes transferring the cell in the cell culture matrix. When transferring the input cell to a cell culture device includes transferring the cell in the cell culture matrix, the cell culture matrix includes sections of at least 1 μL, or at least 5 μL, and may include sections of up to 10 μL, up to 15 μL, up to 25 μL, up to 50 μL, or up to 80 μL.

### Cell Culture Devices

A cell culture device may include, for example, a bioreactor, a spinner flask, and a roller bottom flask. In some embodiments, the cell culture device preferably includes a gas permeable membrane surface. A gas permeable membrane may include a silicone surface including, for example a dimethyl silicone surface. The gas permeable membrane may form any suitable surface of the cell culture device including, for example, a bottom surface or a side of a plate or a flask. In some embodiments, the cell culture device may preferably include a GREX cell culture device (Wilson Wolf Corporation, St. Paul, Minn.).

### Second Cell Culture Medium

In some embodiments, the cell culture device includes a second cell culture medium. In some embodiments, the second cell culture medium may include a feeder-free cell culture medium. In some embodiments, the second cell culture medium may include a serum-free cell culture medium.

In some embodiments, the second cell culture medium includes an iPSC medium. An iPSC medium may include, for example, ESSENTIAL 8 Medium (Thermo Fisher Scientific, Waltham, Mass.), ESSENTIAL 6 Medium (Thermo Fisher Scientific, Waltham, Mass.), or mTeSR1 (StemCell Technologies, Vancouver, Canada).

In some embodiments, the second cell culture medium includes a neural medium. A neural medium may include, for example, DMEM, DMEM F-12, etc.

In some embodiments, the second cell culture medium includes at least one neural induction factor and/or neural growth factor. Neural induction factors and/or neural growth factors may include, for example, N2, B27, fibroblast growth factor (also known as bFGF, FGF2 or FGF-β), transforming growth factor beta (TGFβ), insulin, ascorbate, glutamate, etc.

Without wishing to be bound by theory, it is believed that the methods described herein may allow for the development of input cells into organoids without the addition of a neural induction factor and/or neural growth factor (including, for example, by using ESSENTIAL 6 Medium), providing less expensive and less variable organoid production.

### Cell Culture Process in Cell Culture Device

Once transferred to the cell culture device, the cells may be cultured under any suitable conditions. For example, in some embodiments, the cells may be cultured at a temperature in the range of 32° C. to 40° C. In some embodiments, the cells may be cultured at 37° C. In some embodiments, the cells may be cultured at room temperature (e.g., a temperature in a range of 20° C. to 25° C.). In some embodiments, the cells may be cultured under hypoxic conditions. Hypoxic conditions, as used herein, refer to an environment having less than 20% oxygen.

The second cell culture medium may be changed as required to maintain cell growth. In some embodiments, the cells may be passaged every 3-4 days.

In some embodiments, the culture process may include periodically detaching the cells and/or organoids from a surface of the flasks. For example, the method may include removing the cell and/or organoid from a gas permeable membrane surface at least once during cell culture.

In some embodiments, including when the cell culture medium includes a cell culture matrix, the cell culture matrix may be removed from the organoids using mechanical methods (e.g., with tweezers, a scalpel, and/or forceps) and/or by enzymatic methods (e.g., using hyaluronidase and/or chitosanase). In some embodiments, the cell culture matrix may be removed using the Cell Retrieval Kit from BRTI Life Sciences (Two Harbors, Minn.). In some embodiments, the cell culture matrix may disintegrate, making removal unnecessary.

The cells may be cultured in the cell culture device for at least 1 day, at least 2 days, at least 3 days, at least 4 days, at least 7 days, at least 2 weeks, at least 3 weeks, at least 1 month, at least 2 months, or at least 3 months. In some embodiments, the cells may be cultured for up to 6 months, up to 1 year, or up to five years. In some embodiments, organoids may form after 3 days, after 5 days, after 7 days, after 2 weeks. In some embodiments, A9 neurons may be present in the organoid after at least 7 days, after at least 2 weeks, after at least 3 weeks, after at least 1 month, after at least 6 weeks, after at least 2 months, or after at least 3 months.

### Cell Dis-Aggregation

In some embodiments, cells of the organoids may be dis-aggregated to produce a population of individualized cells. In some embodiments, the cell of the organoid may be dissociated by chemical and/or mechanical dissociation. For example, in some embodiments, the cells may be treated with trypsin and/or EDTA. In some embodiments, the cells may mechanically dissociated using a pipette.

In some embodiments, the dis-aggregated organoid-derived cells may be further cultured. For example, as described in Example 2, the dis-aggregated cells may be plated on MATRIGEL-coated coverslips. In some embodiments, the dis-aggregated cells may be cultured in Neurobasal Medium (Thermo Fisher Scientific, Waltham, Mass.). In some embodiments, the Neurobasal Medium may include a B-27 supplement (Thermo Fisher Scientific, Waltham, Mass.).

### Features of the Midbrain Organoid

The methods described herein may be used to produce organoids including, for example, a midbrain organoid that includes an A9 neuron. In some embodiments, the midbrain organoid may also include an A10 neuron.

In some embodiments, the midbrain organoid preferably includes at least one of a cell expressing glial fibrillary acidic protein (GFAP); a cell expressing microtubule associated protein 2 (MAP2); and a cell expressing myelin basic protein (MBP).

In some embodiments, the midbrain organoid includes at least one of an oligodendrocyte, an astrocyte, and a polydendrocyte. In some embodiments, an oligodendrocyte may be identified by its expression of MBP. In some embodiments, an astrocyte may be identified by its expression of GFAP. In some embodiments, a polydendrocyte may be identified by its expression of chondroitin sulfate proteoglycan 4 (CSPG4).

In some embodiments, a cell of the midbrain organoid may exhibit typical neuronal electrophysiology. Neuronal electrophysiology may be measured by any suitable method including, for example, by patch clamp analysis.

In some embodiments, the presence of an A9 neuron is characterized by expression of at least one of tyrosine hydroxylase, Girk2, and Nurr1. For example, in some embodiments, the presence of an A9 neuron is characterized by expression of tyrosine hydroxylase and Girk2.

In some embodiments, the presence of an A10 neuron is characterized by the expression of at least one of tyrosine hydroxylase, calbindin 1 (CALB1), and Nurr1. For example, in some embodiments, the presence of an A10 neuron is characterized by expression of tyrosine hydroxylase and CALB1.

In some embodiments, the midbrain organoid includes a cell exhibiting expression of at least one of nucleostemin (GNL3), SOX1, SOX2, β-3 tubulin (TUBB3), and nestin (NES). In some embodiments, expression of at least one of GNL3, SOX1, SOX2, TUBB3, and NES may indicate the presence of a neural stem/progenitor cell.

In some embodiments, the midbrain organoid includes a cell exhibiting expression of at least one of nuclear receptor subfamily 4 group A member 2 (NR4A2); LIM homeobox transcription factor 1 alpha (LMX1A); forkhead Box A2 (FOXA2); and orthodenticle homeobox 2 (OTX2). In some embodiments, expression of at least one of NR4A2, LMX1A, FOXA2, and OTX2 may indicate the presence of a dopaminergic neuron progenitor.

In some embodiments, the midbrain organoid includes a cell exhibiting expression of at least one of tyrosine hydroxylase (TH); torsin family 1 member A (TOR1A); corin, serine peptidase (CORIN); and dopa decarboxylase (DDC). In some embodiments, expression of at least one of TH, TOR1A, (CORIN, and DDC may indicate the presence of a dopaminergic neuron.

In some embodiments, the midbrain organoid includes a cell exhibiting expression of potassium voltage-gated channel subfamily J member 6 (KCNJ6). In some embodiments, expression of KCNJ6 may indicate the presence of an A9 neuron.

In some embodiments, the midbrain organoid includes a cell exhibiting expression of calbindin 1 (CALB1). In some embodiments, expression of CALB1 may indicate the presence of an A10 dopaminergic neuron.

In some embodiments, the expression of a marker that indicates a cell type may be measured by detecting protein expression and/or by detecting gene expression. Protein expression and/or gene expression may be detected using any suitable method or combination of methods. For example, expression may be detected by a technique including, for example, immunohistochemical (IHC) staining, immunofluorescence, quantitative Western blot, flow cytometry, RNA-Seq gene expression analysis, quantitative RT-PCR, mass spectroscopy, microarray analysis, etc. In some embodiments, methods of detecting protein expression may be preferred for determining whether a protein is present in a cell because it is possible for an RNA to be expressed but not transcribed into a protein.

Uses for the Cells and/or Organoids

In another aspect, this disclosure describes using the cells (e.g., A9 neurons) and/or organoids described herein for an experimental or therapeutic use. For example, in some embodiments, the cells and/or organoids may be used in drug discovery, to determine how cells interact within an organ, to study the uptake of nutrients, as a cellular model of human disease, etc. In some embodiments, the cells and/or organoids may be used as in a model of a brain disorder. In some embodiments, the cells and/or organoids may be used as a source of a therapeutic cell for the treatment of a brain disorder. A brain disorder may include for example, a neurodegenerative disease such as Alzheimer's Disease or Parkinson's Disease; a genetic brain disorder such as the mucopolysaccharidoses, childhood cerebral adrenoleukodystrophy, and Gaucher's disease; and a brain injury caused by ischemia, stroke, and/or trauma. In some embodiments, of a therapeutic cell may include at least one of a neuron, an oligodendrocyte, and an astrocyte.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Materials

### Passaging Solution/Citrate Buffer (Solution of 0.3 M Potassium Chloride and 0.015 M Sodium Citrate-dihydrate)

The iPSC lines used are described in Table 1.

TABLE 1

| iPS | Cell | | | | |
|---|---|---|---|---|---|
| Line | Line | Sex | Derived cell type | Delivery Method | Reprogramming Factors |
| R58 | ALD1 | M | keratinocytes | Retrovirus | OCT4, SOX2, KLF4, c-MYC |
| | | | | | (Addgene) |
| R76 | ALD2 | M | fibroblasts | Retrovirus | OCT4, SOX2, KLF4, c-MYC |
| | | | | | (Addgene) |
| R77 | ALD3 | M | keratinocytes | Cytotune 2.0 | polycistronic Klf4-Oct3/4-Sox2, |
| | | | | | cMyc, and Klf4. |
| CS1 | WT1 | F | fibroblasts | Retroviral | OCT4, SOX2, KLF4, c-MYC |
| | | | | | (Addgene) |
| CEC | WT2 | M | corneal epithelial | Cytotune 1.0 | Oct3/4, Sox2, Klf4, and cMyc |
| D12-9 | WT3 | F | peripheral blood | Cytotune 1.0 | Oct3/4, Sox2, Klf4, and cMyc |
| GRIPS | WT4 | M | foreskin fibroblasts | Cytotune 1.0 | Oct3/4, Sox2, Klf4, and cMyc |
| CBB | — | F | cord blood | Retroviral | OCT4, SOX2, KLF4, c-MYC |
| 1024 | — | M | bone marrow | Sendai virus | OCT4, SOX2, KLF4, c-MYC |

### Methods

### Cell Culture

1. Add 50 mL ESSENTIAL 8 media (Catalog No. A151700, Thermo Fisher Scientific, Waltham, Mass.) to GREX 100 cell culture device (Catalog No. 800500S, Wilson Wolf Corporation, St. Paul, Minn.) and set aside.
2. Wash 3 T-175 flasks containing iPSCs one time with PBS (17.5 mL each).
3. Add 17.5 mL Passaging Solution/Citrate Buffer into each flask. Wait and observe for 5 minutes or until cells begin to lift off.
4. Aspirate off Passaging Solution/Citrate buffer.
5. Wash cells off of each T-175 flask with 10 mL DMEM/F12 (Thermo Fisher Scientific, Waltham, Mass.) and collect cells into a 50 mL conical tube. Total volume comes to 30 mL.
6. Optionally, rinse all flasks with additional 10 mL DMEM/F12. (Total volume is 40 mL.)
7. Spin in centrifuge for 5 minutes at 150 g/1200 RPM.
8. Aspirate supernatant and resuspend cell pellet in 250 μL of Cell-Mate3D hydration fluid (Catalog No. CM-1001, BRTI Life Sciences, Two Harbors, Minn.).
9. Add hydration fluid mixture to dry blend while vortexing, according to manufacturer's protocol.
10. Transfer Cell-Mate3D to funnel apparatus, according to manufacturer's protocol.
11. Centrifuge to 2700 rpm and stop, according to manufacturer's protocol.
12. Use scalpel to slice small pieces (10 μL to 30 μL) of CellMate and add to prepared Wilson Wolf flask.
13. Culture cells in a 37° C. incubator (5% CO₂, 20% 02); change media every 3-4 days.

### Histology and Immunohistochemistry and Immunocytochemistry

Histology and Immunohistochemistry and Immunocytochemistry were performed as described in Lindborg et al. Stem Cells Translational Medicine, 2016, 5(7):970-979.

### Gene Expression Analysis and Bioinformatics

Organoids including 3 biological replicates from each of 2 different input iPS cell lines were collected at week 6 and lysed in RLT buffer (Qiagen, Venlo, The Netherlands) and stored at −80° C. until processed. RNA was isolated from cell lysates using the RNA mini plus kit (Qiagen) according to manufacturer's instructions. RNAseq (HiSeq, Illumina, San Diego, Calif.) gene expression analysis was performed at University of Minnesota Genomics Center. An established analysis pipeline developed and maintained by the University of Minnesota Informatics Institute (UMII) was used to analyze the raw sequence data. The detailed methods are available on the world wide web at bitbucket.org/jgarbe/gopher-pipelines/wiki/Home. Briefly, the pipeline first performs quality control and adapter trimming using FastQC and Trimmomatic, respectively, and then uses HISAT2 for reads alignment. Finally, the transcript abundance was estimated using Cufflinks and SubRead.

### Results

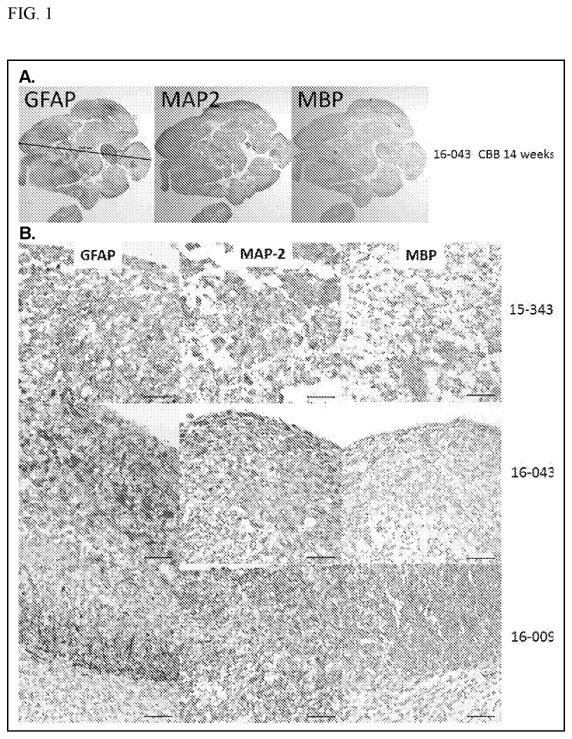

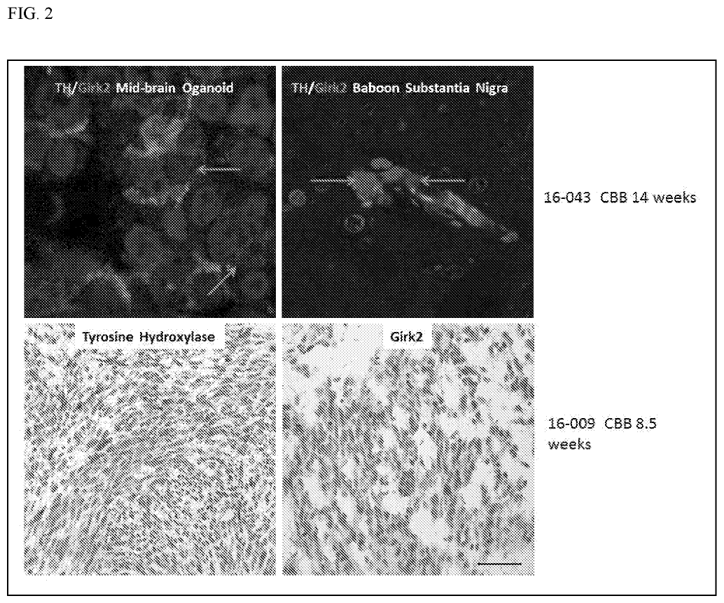

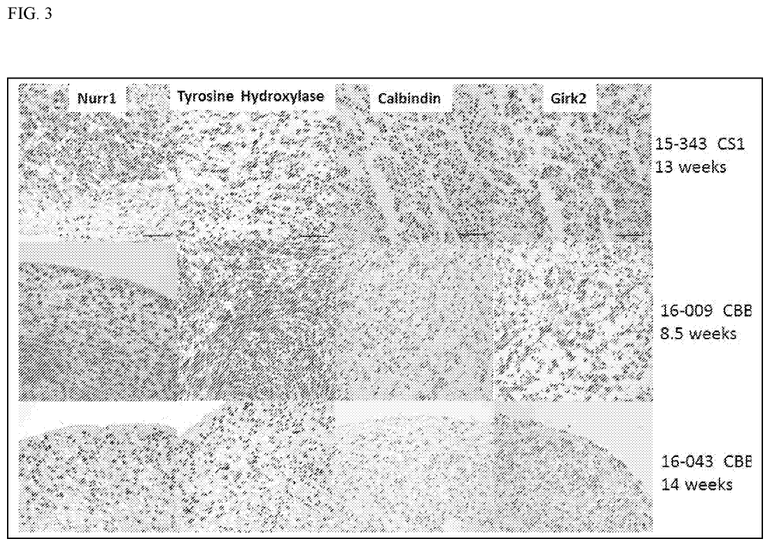

Histologic analysis of organoids between 8.5 weeks and 14 weeks in culture showed extensive regions of neural tissue development in the organoids. Immunohistochemical stains in this time frame showed evidence of development of characteristic brain cell lineages including mature neurons (MAP2), oligodendrocytes (MBP), and astrocytes (GFAP) (FIG. 1). At these time points, there was also consistent IHC evidence characteristic of midbrain dopaminergic neurons with IHC labeling for tyrosine hydroxylase, Girk2, and Nurr1 (FIG. 2 & FIG. 3). Furthermore, specification of A9 nigral dopaminergic neurons was confirmed by the presence of tyrosine hydroxylase (TH)/Girk2 double immunofluorescent positive neurons (FIG. 2).

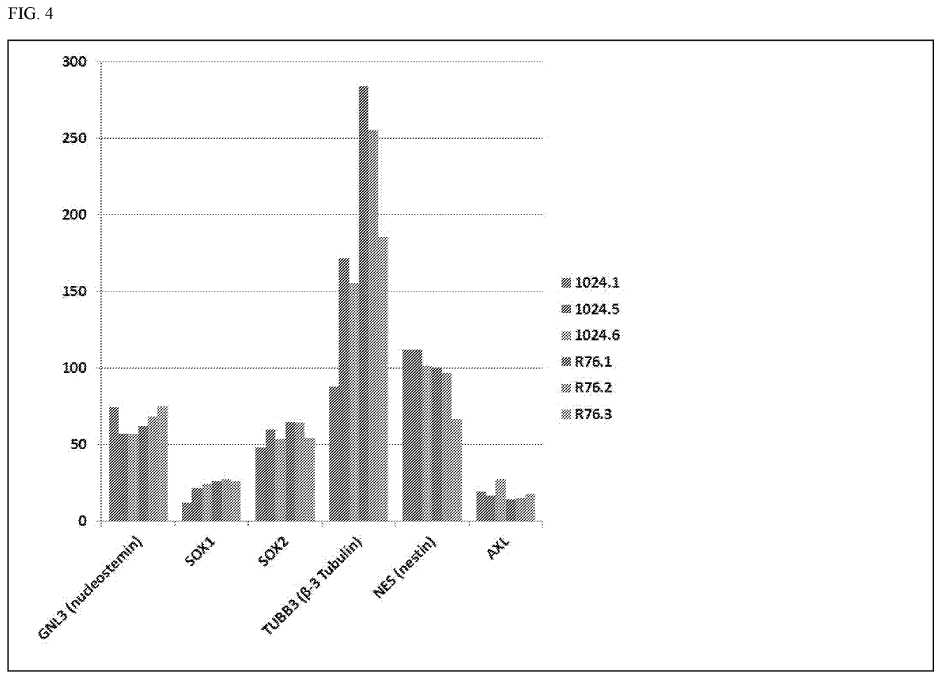

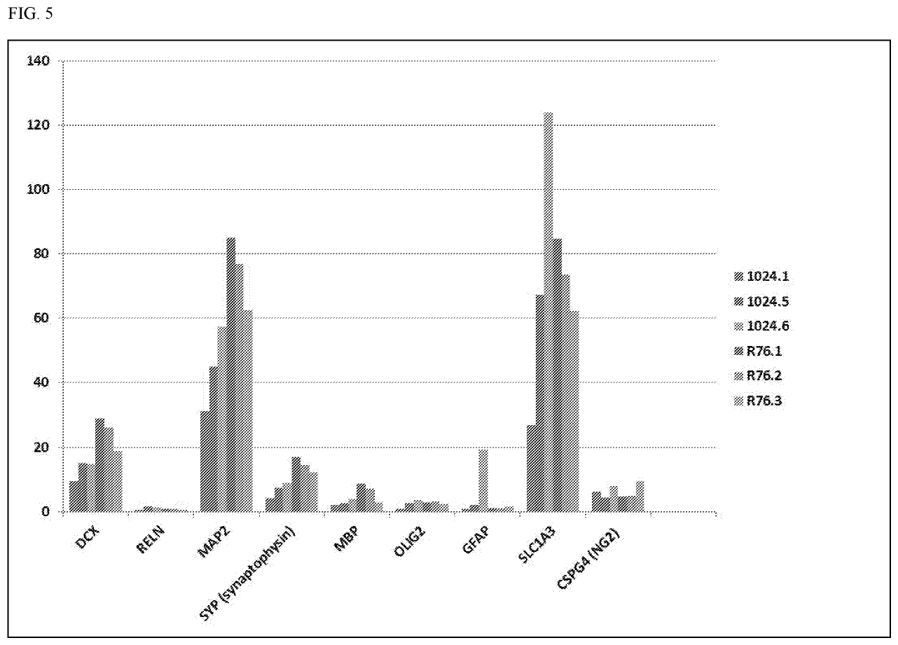

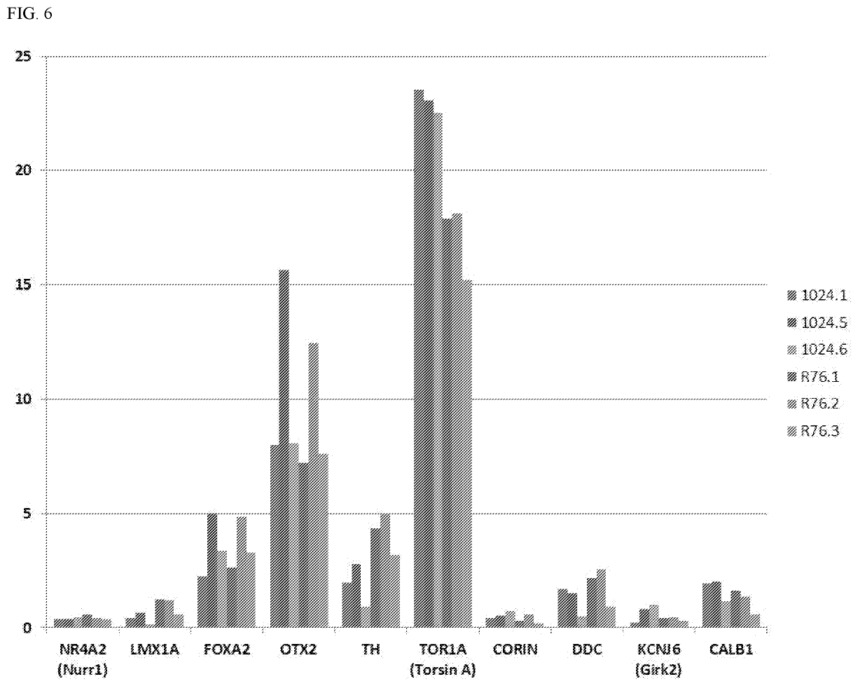

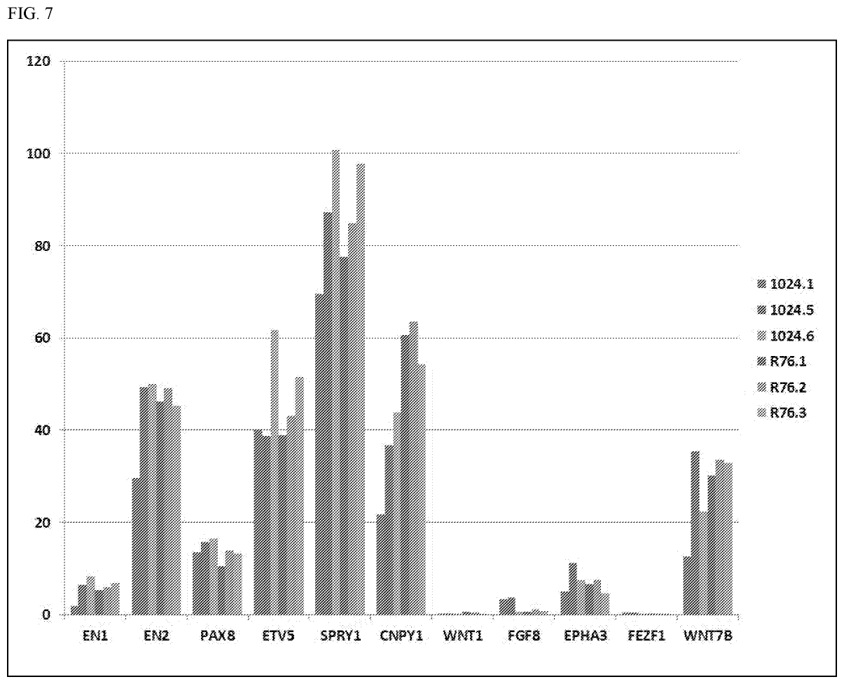

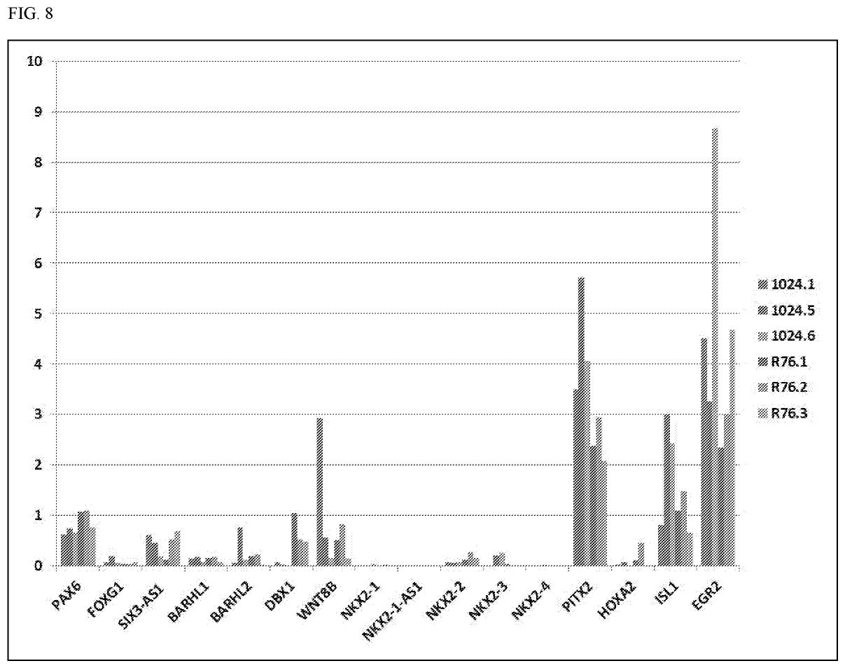

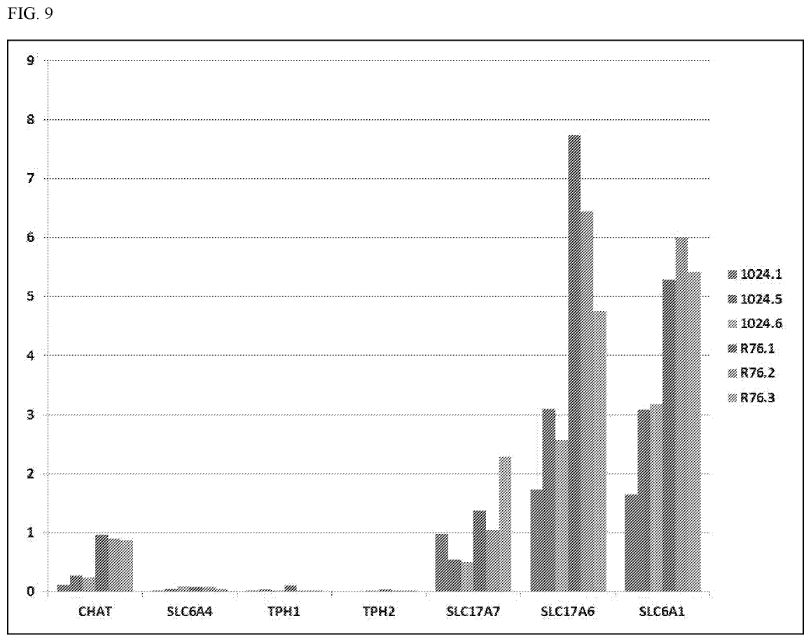

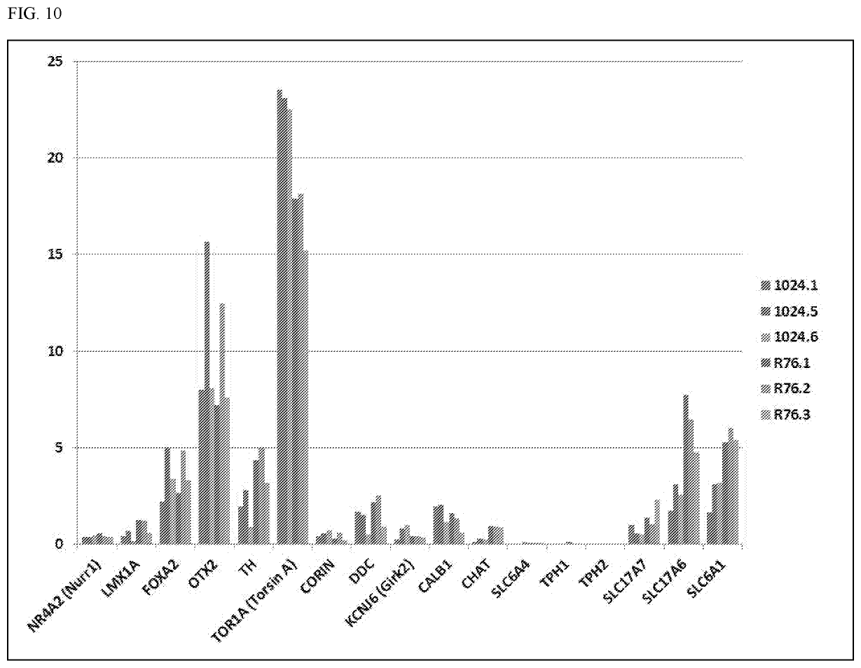

Organoids at 5-6 weeks in culture were further analyzed for global gene expression using whole transcriptome shotgun sequencing analysis (RNA-Seq). In this time frame the organoids showed prominent expression of gene markers for neural stem/progenitor cells as well as for the gene coding for the Zika virus receptor protein (FIG. 4). There was also evidence of expression of gene markers for major brain cell lineages including neurons, oligodendrocytes, astrocytes, and polydendrocytes (FIG. 5). Relatively high expression of dopaminergic neuron markers characteristic of a midbrain phenotype were prominently demonstrated and included markers for both A9 (Girk2) and A10 (calbindin) dopaminergic neurons (FIG. 6 & FIG. 10). Organoids also showed moderate to high levels of expression of gene markers previously shown to be associated with positive engraftment outcomes for neural cell transplants to treat rodents with induced Parkinsonism and relatively lower levels of gene expression for markers associated with negative outcomes (FIG. 7). Organoids showed little expression of gene markers for brain regions outside of the caudal midbrain (A9 dopaminergic neurons are located in caudal midbrain) including forebrain, diencephalon, or rostral midbrain markers and low to moderate expression of markers for hindbrain (a region just caudal to the caudal midbrain) (FIG. 8). Additionally, gene expression markers at low to moderate levels were also found for cholinergic, glutaminergic, and gamma-amino butyric acid (GABAergic) neurons, with little or no expression of gene markers for serotonergic neurons (an undesirable cell type for mid-brain transplants) (FIG. 9).

### Example 2

Organoids were produced from CS1 cells using the methods of Example 1 and cultured for 5 months. Organoids were then dis-aggregated to produce a population of individualized cells as follows: Organoids were rinsed in PBS then treated with 2 mL 0.05% Trypsin-EDTA (Life Technologies; Carlsbad, Calif.) for 2 minutes at 37° C. An additional 2 mL Trypsin-EDTA supplemented with 400 μg DNase1 (Millipore-Sigma, Burlington, Mass.) was added and the cells were mechanically dissociated using a P1000 pipette. The organoids were then incubated for 5 minutes at 37° C. after which, cells were mechanically dissociated using a 1 cc syringe plunger over a 100 μm filter (BD Biosciences; San Jose, Calif.) washing with cold Hank's Balanced Salt Solution (HBSS; Life Technologies, Carlsbad, Calif.) to bring the final volume to 25 mL. The cells were centrifuged at 350×G for 3 minutes at 4° C. The resulting supernatant was removed and cell pellet resuspended in 1 mL cold HBSS for counting using a hemocytometer. The cells were centrifuged a third time and resuspended at a concentration of roughly 5×10⁴ cells per μL of cold HBSS. The final cell solution was counted and viability was assessed using a Trypan Blue exclusion method. The final cell count was calculated as the total number of viable cells per μL. The organoid-derived cell population was then plated on MATRIGEL-coated coverslips and cultured in Neurobasal Medium (Thermo Fisher Scientific, Waltham, Mass.) with B-27 supplement (Thermo Fisher Scientific, Waltham, Mass.).

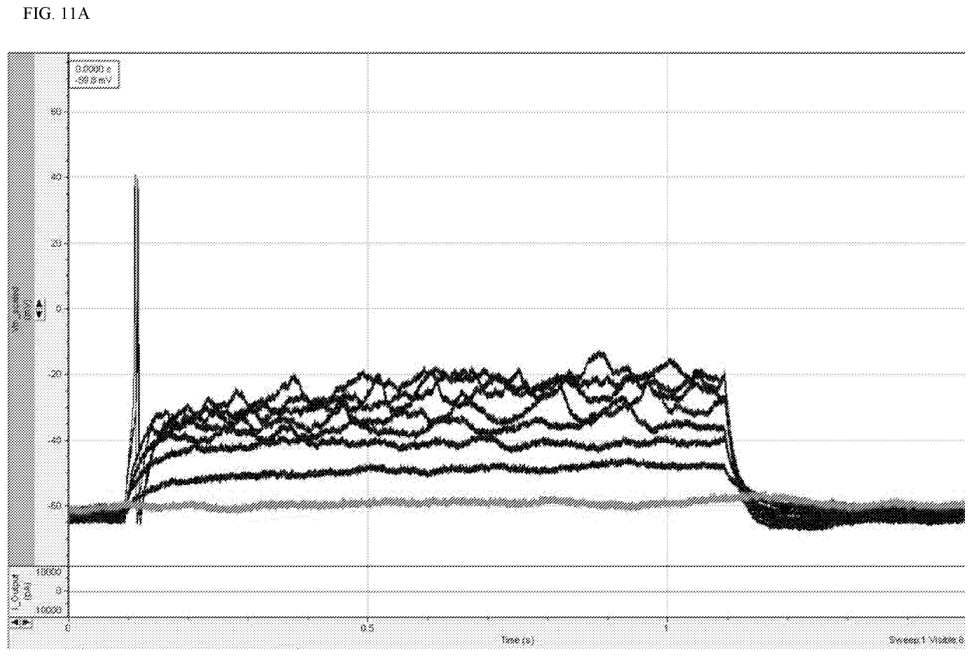

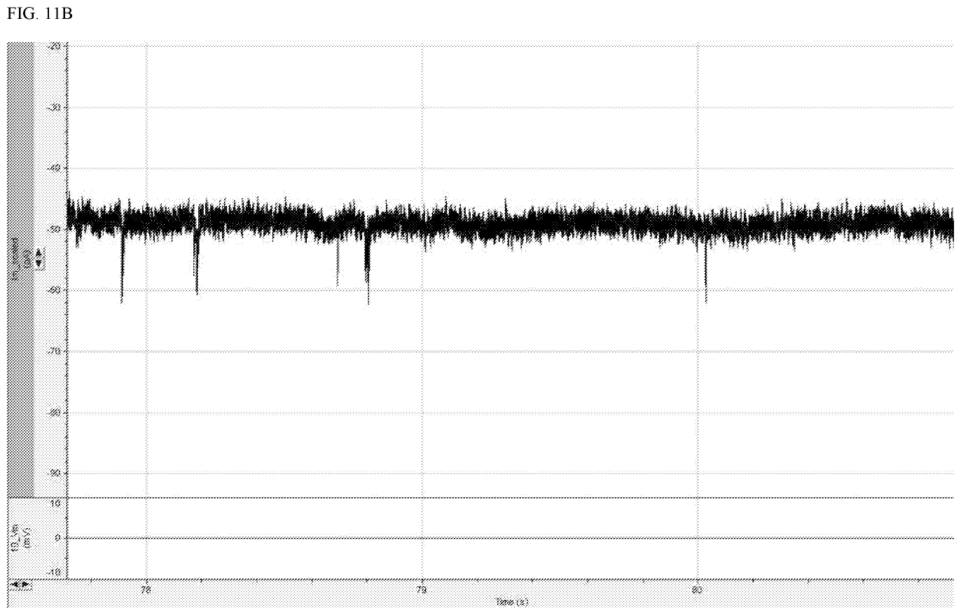

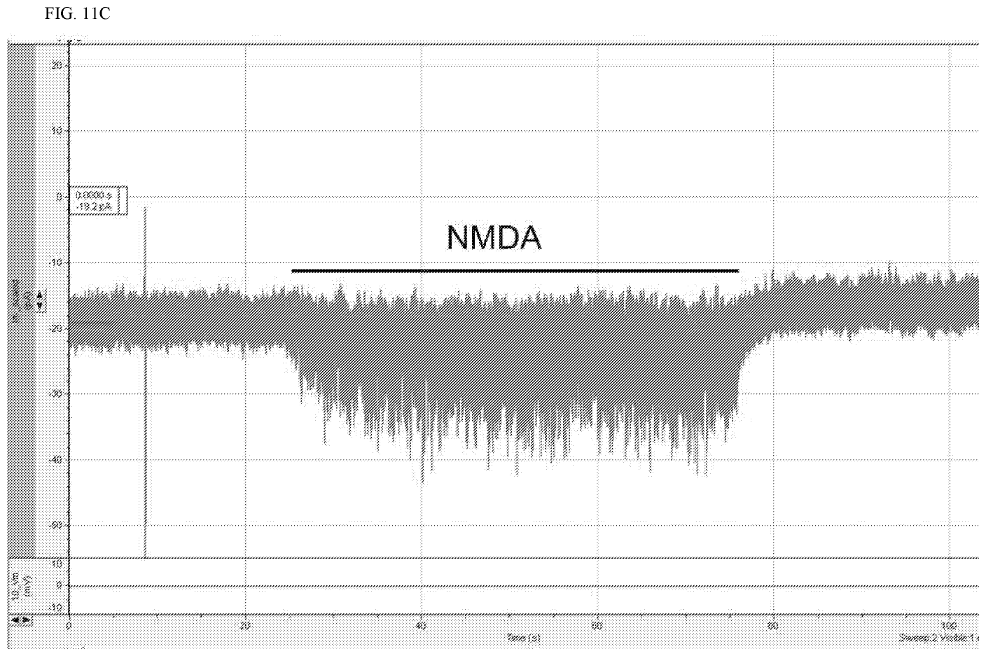

A patch-clamp study of these cell preparations was conducted; results are shown in FIG. 11. FIG. 11A shows a single neuron's (normal) response to electrical stimulation. FIG. 11B shows spontaneous electrical activity of a neuron indicating that it is in contact with other neurons which are stimulating it to respond. FIG. 11C shows neuron responses to NMDA indicating the presence of glutaminergic neurons.

### Example 3

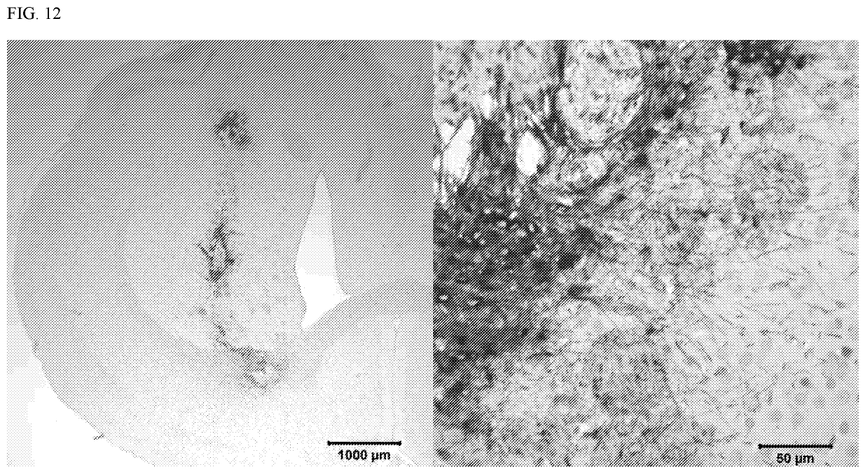

Nude rat brain (striatum) transplanted with 300,000 cells derived from 10-week organoids produced from CS1 or 1024 cells using the methods of Example 1 and using the organoid dis-aggregation method in Example 2. Four months later, tissue sections were prepared and stained for human-specific STEM121; results are shown in FIG. 12. Robust engraftment of the cells at four months post-transplantation was observed.

The complete disclosure of all patents, patent applications, and publications, and electronically available material (including, for instance, nucleotide sequence submissions in, for example, GenBank and RefSeq, and amino acid sequence submissions in, for example, SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A method comprising:
introducing an input cell into a cell culture medium comprising hyaluronic acid, wherein the cell culture medium comprises a cell culture matrix;
transferring the input cell to a cell culture device, wherein the cell culture device comprises a bioreactor comprising a gas permeable membrane surface;
culturing the cell in the cell culture device for at least 7 days; and
producing a midbrain organoid comprising an A9 neuron.

2. The method of claim 1, wherein the input cell comprises an embryonic stem cell, an induced pluripotent stem cell, or a neural progenitor cell.

3.-5. (canceled)

6. The method of claim 1, wherein introducing the cell into the cell culture matrix comprises introducing a single cell or introducing a colony of cells.

7. (canceled)

8. The method of claim 1, wherein introducing the input cell in the cell culture matrix comprises introducing an embryoid body.

9. The method of claim 1, wherein transferring the input cell to a cell culture device comprises transferring the cell in the cell culture matrix.

10. The method of claim 9, wherein at the time of transferring the input cell to a cell culture device, the cell culture matrix comprises sections of up to 80 μL.

11.-15. (canceled)

16. The method of claim 9, wherein at the time of transferring the input cell to a cell culture device, the input cell is present in the cell culture matrix at a concentration of at least 7.6×10⁵ cells per 10 μL matrix.

17.-19. (canceled)

20. The method of claim 1, wherein the cell culture device comprises a second cell culture medium.

21. The method of claim 20, wherein the second cell culture medium comprises a serum-free cell culture medium, a feeder-free cell culture medium, an iPSC medium, or a neural medium, or a combination thereof.

22.-24. (canceled)

25. The method of claim 20, wherein the second cell culture medium comprises a neural induction factor, or a neural growth factor, or both.

26.-30. (canceled)

31. The method of claim 1, the method further comprising removing the cell from the gas permeable membrane surface.

32.-34. (canceled)

35.-37. (canceled)

38. The method of claim 1, the midbrain organoid comprising
a cell expressing glial fibrillary acidic protein (GFAP);
a cell expressing microtubule associated protein 2 (MAP2); or
a cell expressing myelin basic protein (MBP);
or a combination thereof.

39. The method of claim 1, the midbrain organoid comprising an oligodendrocyte, an astrocyte, or a polydendrocyte, or a combination thereof.

40.-45. (canceled)

46. The method of claim 1, the midbrain organoid comprising an A10 neuron.

47.-49. (canceled)

50. The method of claim 1, the method further comprising dis-aggregating the cells of the midbrain organoid to produce a population of individualized cells.

51. The method of claim 50, the method further comprising culturing a cell from the population of individualized cells.

52. A midbrain organoid or a cell of a midbrain organoid generated using the method of claim 1.

53. A method of using the cell of the midbrain organoid or the midbrain organoid of claim 52 for a therapeutic use.

54. The method of claim 53, the method comprising using the cell of the midbrain organoid as a therapeutic cell for the treatment of a brain disorder or in a model of a brain disorder.

55.-59. (canceled)

60. A method of using the cell of the midbrain organoid or the midbrain organoid of claim 52 for an experimental use.
